(19) United States
(12) Patent Application Publication
(10) Pub. No.: US 2026/0159811 A1
(43) Pub. Date: Jun. 11, 2026

(54) METHODS AND COMPOSITIONS FOR PRODUCTION OF CULTURED MEAT

(71) Applicant: ATELIER MEATS CORP., Calgary (CA)

(72) Inventors: Maghsoud DARIANI, Fanwood, NJ (US); Mohit BHATIA, Manalapan, NJ (US)

(21) Appl. No.: 18/708,043

(22) PCT Filed: Nov. 2, 2022
(86) PCT No.: PCT/US2022/048680

§ 371 Date: May 7, 2024

Related U.S. Application Data
(60) Provisional application No. 63/276,795, filed on Nov. 8, 2021.

(51) Int. Cl. C12N 5/077; A23L 13/00; C12N 5/0775

(52) U.S. Cl. CPC C12N 5/0656; A23L 13/00; C12N 5/0662; C12N 5/0668; C12N 2533/50; C12N 2533/52; C12N 2533/54; C12N 2533/90

(57) ABSTRACT

Compositions of decellularized nonhuman mammalian placenta and scaffolds thereof for use in production of cultured meat fit for human consumption are provided.

This patent application claims the benefit of priority from U.S. Provisional Application Serial No. 63/276,795, filed Nov. 8, 2021, the content of which is incorporated by reference in its entirety.

### FIELD

The present disclosure relates to compositions and scaffolds of decellularized nonhuman mammalian placenta and methods for their production and use in production of cultured meat.

### BACKGROUND OF INVENTION

Cultured or cultivated meat is meat that is generated in a laboratory by culturing cells on a suitable scaffold. The cell types used are either primary cells or stem cells taken from the animal of interest. These cells are grown in bio-reactors to an amount that can be used to populate a scaffold. Each cell type typically requires its own specialized media for growth, which includes several growth factors and cytokines in addition general media components.

The scaffold is important in that it must allow for the attachment followed by proliferation of the various cell types, and formation of the cultured meat. Most commonly synthetic or plant based scaffolds are used. These scaffolds either remain a part of the edible cultured meat product or are resorbed during the production. As such, meat products generated are soft meat products similar to nuggets or burger patties. A hard bone like surface that is amenable to cell attachment is necessary to create steak-like meats.

Decellularized human placental scaffolds have been disclosed (Bhatia et al. Wounds. 2008 20 (2): 29-36; Published U.S. Patent Application No. 2007/0020225). However, the methods used lead to scaffolds that are devoid of key cell-adhesion proteins and growth factors necessary for the construction of a cell-scaffold combination product. Further, the structure of bovine or porcine placentas are distinct from human placentas (Kakabadzi, A and Kakabadzi, Z (2015) Prospect of Using Decellularized Human Placenta and Cow Placentome for Creation of New Organs: Targeting the Liver (Part I: Anatomic Study).

There is a need for natural scaffolds that can be used in the culturing of nuggets and patties as well as steak-like cultured meats to which cells easily attach and proliferate and which allows for cell-to-cell interactions resulting in production of their own growth factors, thereby reducing the need for externally added growth factors and cytokines.

### SUMMARY

An aspect of this disclosure relates to compositions comprising decellularized nonhuman mammalian placenta with an intact extracellular matrix and/or vasculature.

Another aspect of this disclosure relates to methods for producing the compositions of decellularized nonhuman mammalian placenta by exposing the nonhuman mammalian placenta to alternating concentrations of high and low sodium chloride, a detergent and/or one or more proteolytic enzymes, and/or elevating the pH of the nonhuman mammalian placenta to greater than 8.0 via addition of a base.

Another aspect of this disclosure relates to scaffolds comprising the decellularized nonhuman mammalian placenta composition which allow for attachment and proliferation of muscle cell precursors and other cell types critical to formation of cultured meat.

Another aspect of this disclosure relates to methods for culturing nonhuman mammalian cells into cultured nonhuman mammalian meat. In these methods, one or more nonhuman mammalian cells selected from stem cells, cytoskeletal cells, muscle cells, adipocytes, endothelial cells, fibroblasts and keratinocytes are seeded onto a scaffold comprising the decellularized nonhuman mammalian placenta composition and grown in culture media under conditions in which the cells form a nonhuman mammalian meat.

Yet another aspect of this disclosure relates to use of the compositions comprising decellularized nonhuman mammalian placenta and scaffolds thereof in production of nonhuman mammalian cultured meat for consumption.

### BRIEF DESCRIPTION OF FIGURES

FIG. 1 shows cotyledons dissected from the placentome and collected during isolation of placental ECM using a freeze-drying step to accelerate decellularization.

FIG. 2 shows collagen content tested using the hydroxyproline content method in the decellularized bovine placental extracellular matrix (ECM).

FIG. 3 shows elastin content in the decellularized bovine placental extracellular matrix (ECM).

FIG. 4 shows results of immunostaining of fibronectin in fresh and decellularized tissues.

FIG. 5 shows results of testing the placental ECM for its ability to allow for cell attachment which are indicative of bovine placental ECM being a functional substrate for cell attachment and growth.

### DETAILED DESCRIPTION

Various embodiments and aspects of the inventions will be described with reference to details discussed below and will illustrate the various embodiments. The following description of the invention is not to be construed as limiting the invention. Numerous specific details are described to provide a thorough understanding of various embodiments of the present invention. However, in certain instances, well-known or conventional details are not described in order to provide a concise discussion of embodiments of the present inventions. Reference in the specification to “one embodiment” or “an embodiment” or “another embodiment” means that a particular feature, structure, or characteristic described in conjunction with the embodiment can be included in at least one embodiment of the invention. The appearances of the phrase “in one embodiment” in various places in the specification do not necessarily all refer to the same embodiment.

This invention relates to compositions of decellularized nonhuman mammalian placenta and use of these compositions in scaffolds seeded with nonhuman mammalian cells to produce cultured meat fit for human consumption.

In one nonlimiting embodiment, the placenta is taken from the nonhuman mammal after birth of the baby mammal. There is no harm done to the mother or the baby.

The nonhuman mammalian placenta is then decellularized while leaving intact the extracellular matrix and/or vasculature.

In various embodiments, smooth muscle cell precursors, adipocytes, endothelial cells, fibroblasts are isolated from the placenta prior to decellularization. These cells are cultured to sufficient densities to create cell banks for later seeding onto scaffolds as disclosed herein.

In one nonlimiting embodiment, the placenta is decellularized in its whole form.

In another nonlimiting embodiment, the placenta is decellularized after cutting into small pieces.

Various methods for decellularization can be used.

In one nonlimiting embodiment, the intact or cut placental tissue is washed with water and/or rinsed repeatedly with 2 M sodium chloride and water to remove blood and blood components.

In one nonlimiting embodiment, the placental tissue is then washed with a mild detergent and sodium peroxide to remove lipids and cellular debris. In one nonlimiting embodiment, the tissue is washed with Triton-X100. In another nonlimiting embodiment, the tissue is washed with Tween-20.

In one nonlimiting embodiment, the placental tissue is washed with a proteolytic enzyme for 24-48 hours. In one nonlimiting embodiment, the proteolytic enzyme is trypsin, chymotrypsin or pepsin or a combination of any of these enzymes.

In one nonlimiting embodiment, the tissue is rinsed with 0.5-2 M sodium chloride with or without detergent to decellularize the placental tissue.

In one nonlimiting embodiment, the placental tissue is freeze-dried prior to use of detergent and/or salt to decellularize the tissue.

In one nonlimiting embodiment, the composition of decellularized nonhuman mammalian placenta comprises at least 50% collagen.

In one nonlimiting embodiment, the composition of decellularized nonhuman mammalian placenta comprises 25% elastin or less.

In one nonlimiting embodiment, the composition of decellularized nonhuman mammalian placenta comprises intact fibronectin, laminin and/or glucosaminoglycans.

In one nonlimiting embodiment, the composition of decellularized nonhuman mammalian placenta comprises growth factors such as, but not limited to, VEGF and FGF.

In one nonlimiting embodiment, the decellularized placental tissue is freeze dried and used intact as a soft scaffold for cultured meat production.

In alternative nonlimiting embodiments, the decellularized placental tissue is ground to a paste and then heat dried to make sheets. The sheets may serve as a scaffold or may be compacted using heat to make solid 3-dimensional scaffolds in the shape of cylinders, cubes or other 3-dimensional forms.

In one nonlimiting embodiment, a scaffold for attachment and proliferation of muscle cell precursors and other cell types critical to formation of cultured meat is produced via 3-dimensional printing of the ground paste into a scaffold.

Scaffolds comprising the decellularized nonhuman mammalian placental tissue as disclosed herein have a unique composition and physical structure with intact extracellular matrix and/or vasculature which allow for attachment and proliferation of muscle cell precursors and other cell types critical to formation of cultured meat suitable for human consumption. Further, it is expected that somatic cells, precursor cells and stem cells from a particular nonhuman mammal will preferentially attach and proliferate on a scaffold of the same nonhuman mammal, e.g. bovine cells on a scaffold of decellularized bovine placental tissue, porcine cells on a scaffold of decellularized porcine placental tissue, ovine cells on a scaffold of decellularized ovine placental tissue, caprine cells on a scaffold of decellularized caprine placental tissue, etc. Co-culturing of various cell types of a particular nonhuman mammal on a scaffold of the same nonhuman mammal is also expected to reduce the need for extraneously added growth factors and cytokines.

Thus, by the phrases “nonhuman mammal” or “nonhuman mammalian” is it meant to encompass any mammal from which humans can derive meat from for consumption. Nonlimiting examples include cattle, pigs, sheep or lambs, goats, bison, elk and deer.

Preferred cell types for seeding of the scaffolds of this invention are stem cells taken from the nonhuman mammal of interest. Stem cells for use in production of cultured meat can be grown (or cultured) in the laboratory using bio-reactors to high densities, followed by differentiation to somatic cells. However, differentiation to each cell type can require separate concoctions of media components such as growth factors and cytokines to generate those cells. The need to culture several cell types independently can result in a substantial cost contribution to product generation.

With the present invention, scaffolds can be seeded and cultured with one or more cell types to initiate formation of the cultured meat product. In addition to or alternatively to stem cells, scaffolds may be seeded with one or more nonhuman mammalian cells selected from cytoskeletal cells, muscle cells, adipocytes, endothelial cells, fibroblasts and keratinocytes.

Selected cell types may be added in specific order to build the cultured meat product. Growth factors may be added primarily to stimulate the first cell type added. The first cell type added is expected to produce growth factors and cytokines to stimulate the subsequently added cell type.

This reduces the need to add growth factors and cytokines for each cell type added. For example, fibroblasts may be added first. Fibroblasts produce VEGF (Vascular Endothelial Growth Factor). VEGF stimulates endothelial cells to form blood vessels. In turn, they stimulate other cells to release other cytokines and growth factors. By stimulating cells to produce their own signals, the need to add large amounts of many growth factors and cytokines is circumvented. The use of reduced amount of growth factors and cytokines has economic implications as well.

Further, by modifying media conditions and cell types for culturing, the desired composition of the cultured meat product can be altered to a composition 4 different fat, carbohydrate and protein concentrations or percentages.

Once seeded, the scaffolds can be cultured in a fed-batch process and/or continuous stir tanks and/or cell culture bags.

In one nonlimiting embodiment, the seeded scaffolds are cultured in cell culture media comprising VEGF.

The following nonlimiting examples are provided to further illustrate the present invention.

### EXAMPLES

### Example 1: Isolation of Extracellular Matrix (ECM) From Bovine Placenta

Bovine placental tissues were collected right after birth and stored at 4° C. prior to processing. Tissues were washed with water to remove blood and other debris.

Individual placentome was cut out and saved in a 50 mL conical tube. Tubes containing the tissues were stored at −20° C. till the decellularization. Decellularization of the placentome tissues was carried out using three different procedures. Specifically, frozen placentome tissues were thawed at room temperature (RT). Tissues (cut into 3 cm×3 cm pieces) were rinsed with dH20 multiple times in a container until no significant blood was in the rinse solution. Rinsed tissues were then disinfected in 70% ethanol in the same container with shaking at RT for 30 minutes. Disinfected tissues were transferred to sterile bottles (500 mL volume size) and washed with sterile water.

For the first procedure, referred to as DOC decell, tissues were immersed in sterile DOC solution (2% deoxycholate+10 mM EDTA+10 mM HEPES in water) (volume ratio of tissue:solution=1:4) and mixed by shaking at RT for overnight (20 hours). The DOC solution was removed and fresh DOC solution was added and mixed for 4 hours. This DOC solution treatment was repeated for another two sets of 20 hours+4 hours.

For the second procedure, referred as SDS decell, tissues were immersed in sterile SDS solution (1% SDS in water) (volume ratio of tissue: solution=1:4) and mixed by shaking at RT for overnight (20 hours). The SDS solution was removed and fresh SDS solution was added and mixed for 4 hours. This SDS solution treatment was repeated for another two sets of 20 hours +4 hours.

For the third procedure, referred to as DOC+SDS decell, tissues were immersed in sterile 2% DOC solution and mixed by shaking at RT for overnight (20 hours). The DOC solution was removed and fresh DOC solution was added and mixed for 4 hours. The DOC solution was removed and the tissues were immersed in sterile 1% SDS solution and mixed by shaking at RT for overnight (20 hour). The SDS solution was removed and fresh SDS solution was added and mixed for 4 hours. The SDS solution treatment was repeated for another 20 hours+4 hours.

All treated tissues were then washed with water three times with shaking (1 hour for each time) followed by washing with water for 20 hours. Decellularized tissues were frozen at −80° C. then thawed at 37° C. to reduce the water retention in decellularized tissues. Decellularized tissues were stored at −20° C. till further analysis.

Multiple procedures such as 2% deoxycholate solution alone (DOC decell), 1% SDS solution alone (SDS decell) and sequentially decellularization with 2% DOC and 1% SDS (DOC+SDS decell) were tested. The decellularized placentome tissues processed using different procedures all had similar structure and texture to that of the fresh tissues.

### Example 2: Isolation of Placental ECM Using a Freeze-Drying Step to Accelerate Decellularization

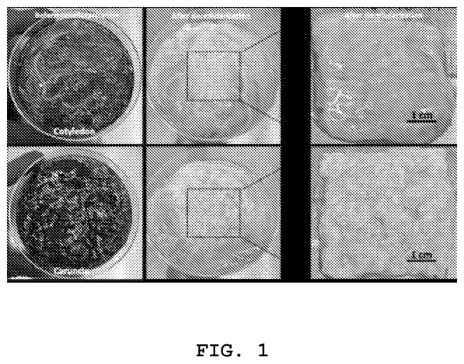

Cotyledons were dissected from the placentome and collected. See FIG. 1. Samples were frozen immediately.

Frozen tissue was freeze-dried for 3 days followed by grinding of the tissue. Ground tissue was added to 50 ml conical tubes which were filled with 50 ml 0.5 M NaCl solution and placed on a rocker tray to shake for 24 hours.

After 2 washes with 50 mL NaCl every 24 hours, the solution was replaced with 50 ml 1% SDS solution and the conical tubes were placed back on the rocker for 24 hours. The SDS solution was next replaced with 50 ml 0.1N sodium hydroxide solution and the conical tubes were placed back on rocker for 24 hours. The sodium hydroxide solution was removed and tissue was washed with PBS. The decellularized tissue was freeze dried for 24-72 hours until completely dry.

### Example 3: Composition of Bovine Placental Extracellular Matrix

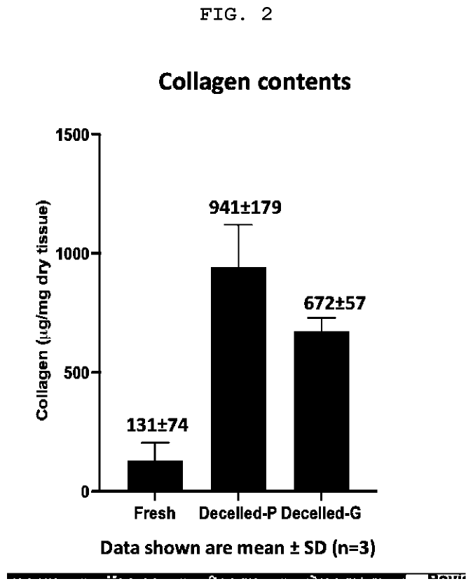

The decellularized bovine placental ECM was tested for the presence of extracellular matrix components including collagen, elastin and fibronectin. Collagen content was tested using the hydroxyproline content method as shown in FIG. 2. The hydroxyproline method involves a digestion of the tissue using the enzyme papain, degradation of the collagen using strong acid at 120° C. and quantification of the hydroxyproline content using Chloramine-T and Ehrlich's solution. The bovine placental ECM contains greater than 90% collagen by dry weight (>90% w/w of dry tissue).

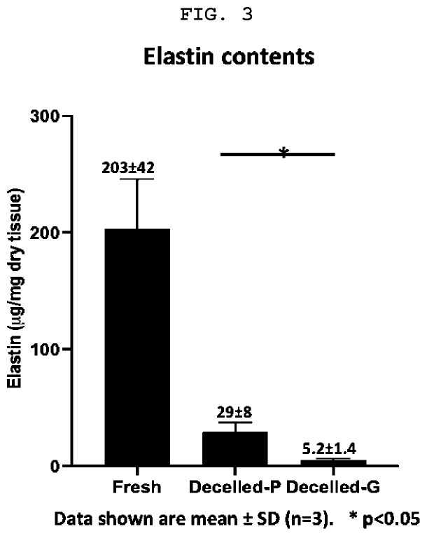

Elastin content was measured using the Elastin assay kit as shown in FIG. 3. This method involves extraction of elastin from the bovine placental ECM using oxalic acid at 100° C. followed by binding to Fastin dye. Elastin content is measured and quantified spectrophotometrically using a UV-VIS spectrophotometer. The elastin content in bovine placental collagen is less than 5% by dry weight (<5% w/w of dry tissue).

### Example 4: Detection of Fibronectin

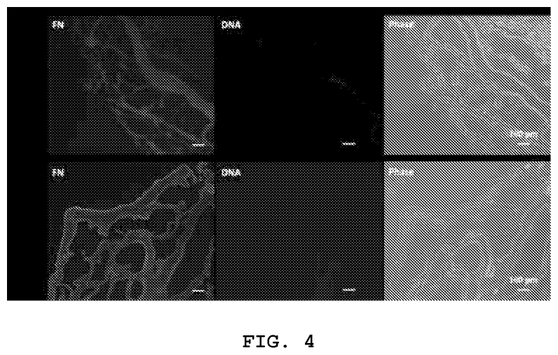

The presence of fibronectin, a key cell adhesion protein was detected using immunostaining methods. Immunostaining of fibronectin in fresh and decellularized tissues is shown in FIG. 4. Fresh and decellularized tissue sections were stained with anti-FN antibody followed by secondary antibody conjugated with Alexa Fluor488. Representative images are shown in FIG. 4. Data shows the presence of fibronectin in bovine placental ECM.

### Example 5: Cell Growth on Bovine Placental ECM

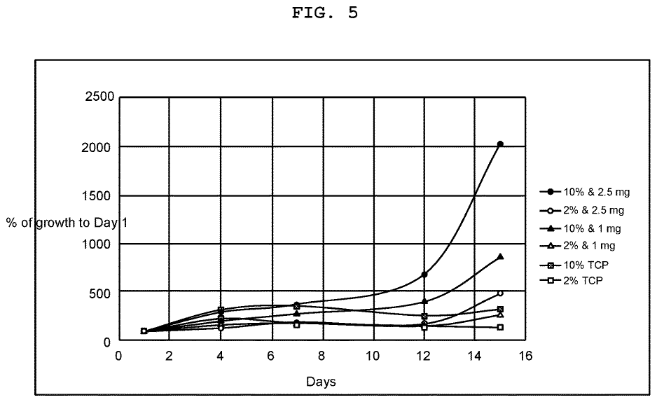

Bovine placental ECM was decellularized as per methods described in Examples 1 and 2. The ECM in its particulate form was attached to tissue culture plates (TCPs). TCPs have the ability to provide adherence to fibroblasts and other stem cell types such as mesenchymal cells and umbilical cord stromal stem cells. To test the placental ECM for its ability to allow for cell attachment, non-adherant TCPs were used and ECM was attached to those TCP wells. Over a period of 15 days, stromal stem cells attached better to the ECM containing plates relative to the TCP plates (FIG. 5). These results show that bovine placental ECM is a functional substrate for cell attachment and growth.

1. A composition comprising decellularized nonhuman mammalian placenta with an intact extracellular matrix and/or vasculature.

2. The composition of claim 1 wherein the decellularized nonhuman mammalian placenta is ground to a paste.

3. The composition of claim 2 wherein the paste is heat dried and formed to make sheets and/or 3-dimensional objects.

4. The composition of claim 1 wherein the decellularized placenta is freeze-dried.

5. The composition of claim 1 which comprises at least 50% collagen.

6. The composition of claim 1 which comprises 25% elastin or less.

7. The composition of claim 1 which comprises intact fibronectin, laminin and/or glucosaminoglycans.

8. The composition of claim 1 which comprises growth factors.

9. The composition of claim 8 wherein the growth factors are VEGF and FGF.

10. A method for producing the composition claim 1, said method comprising decellularizing the nonhuman mammalian placenta via:
exposing the nonhuman mammalian placenta to alternating concentrations of high and low sodium chloride, a detergent and/or one or more proteolytic enzymes; and/or
elevating the pH of the nonhuman mammalian placenta to greater than 8.0 via addition of a base.

11. A scaffold comprising the composition of claim 1.

12. The scaffold of claim 11 which allows for attachment and proliferation of muscle cell precursors and other cell types critical to formation of cultured meat.

13. The scaffold of claim 11 further comprising one or more nonhuman mammalian cells selected from stem cells, cytoskeletal cells, muscle cells, adipocytes, endothelial cells, fibroblasts and keratinocytes.

14. A method for culturing nonhuman mammalian cells into cultured nonhuman mammalian meat, said method comprising seeding a scaffold of claim 11 with one or more nonhuman mammalian cells selected from stem cells, cytoskeletal cells, muscle cells, adipocytes, endothelial cells, fibroblasts and keratinocytes and growing the seeded cells in culture media under conditions in which the cells form a nonhuman mammalian meat.

15. The method of claim 14, wherein the cells are cultured in a fed-batch process and/or continuous stir tanks and/or cell culture bags.

16. The method of claim 14, wherein the cell culture media comprises VEGF.

17. A method for production of nonhuman mammalian cultured meat for consumption, said method comprising culturing one or more nonhuman mammalian cells selected from stem cells, cytoskeletal cells, muscle cells, adipocytes, endothelial cells, fibroblasts and keratinocytes with a composition of claim 1 to produce a nonhuman mammalian culture meat for consumption.

18. A method for production of nonhuman mammalian cultured meat for consumption, said method comprising culturing one or more nonhuman mammalian cells selected from stem cells, cytoskeletal cells, muscle cells, adipocytes, endothelial cells, fibroblasts and keratinocytes on a scaffold of claim 11 to produce a nonhuman mammalian culture meat for consumption.

19. A method for producing a scaffold for attachment and proliferation of muscle cell precursors and other cell types critical to formation of cultured meat, said method comprising 3-dimensionally printing the paste of claim 2 into the scaffold.
